(19) United States
(12) Patent Application Publication
(10) Pub. No.: US 2020/0063178 A1
(43) Pub. Date: Feb. 27, 2020

(54) BIOLOGICAL INDICATOR

(71) Applicant: American Sterilizer Company, Mentor, OH (US)

(72) Inventors: William A. Yirava, Willoughby, OH (US); Jodi R. Alt, Chardon, OH (US); Stephen M. Norris, Mentor, OH (US); Nicholas M. White, Mentor, OH (US)

(21) Appl. No.: 16/106,870

(22) Filed: Aug. 21, 2018

(51) Int. Cl. C12Q 1/22; G01N 31/22

(52) U.S. Cl. CPC C12Q 1/22; C12Q 1/04; G01N 31/226

(57) ABSTRACT

This invention relates to a biological indicator for use with a sterilization process, comprising a carrier on which an inoculum is placed and dehydrated, the inoculum comprising a test organism in the form of bacterial spores and an effective amount of an excipient to enhance recovery of the bacterial spores from the dehydrated inoculum. In an embodiment, the inoculum further comprises a carbohydrate.

### TECHNICAL FIELD

This invention relates to a biological indicator and, more particularly, to a biological indicator in the form of a carrier on which an inoculum is placed and dehydrated, the inoculum comprising test organisms in the form of bacterial spores and an excipient to enhance recovery of the spores from the dehydrated inoculum.

### BACKGROUND

Biological indicators, which typically comprise a carrier and test organisms (e.g., bacterial spores) deposited on the carrier, may be used to monitor sterilization processes, for example, steam and vaporous hydrogen peroxide sterilization processes. The biological indicator is placed in a sterilization chamber and subjected to a sterilant along with the load intended for sterilization (e.g., a medical device). Following the sterilization cycle, the biological indicator is exposed to a recovery media and incubated for the purpose of determining if any of the test organisms are viable. A successful sterilization cycle results in a complete inactivation (no outgrowth) of the test organisms. An unsuccessful sterilization cycle results in an incomplete inactivation (outgrowth detected) of the test organisms.

### SUMMARY

Biological indicators typically comprise standardized preparations of specific test organisms with known characteristics (e.g., a defined population, purity, resistance characteristic, etc.). The test organisms may be microorganisms capable of forming endospores. As such, the test organisms may be in the “spore state,” that is, they may be spores, for example, bacterial spores. A biological indicator may be prepared by depositing an inoculum of the test organisms onto a carrier and then dehydrating the inoculum. The carrier may comprise a substrate, such as filter paper, or it may comprise an interior surface of a self-contained biological indicator (SCBI).

A problem in the art relates to the fact that the inoculum that is used to form the biological indicator is dehydrated or dried, and in order to evaluate the test organisms (i.e., bacterial spores) using a recovery media, the practice is to separate and recover the test organisms from the dehydrated inoculum. In many cases, recovery of the test organisms from the dehydrated inoculum is not sufficient to provide desired test results for evaluating the effectiveness of the sterilization due to the fact that many of the test organisms tend to remain bound to the carrier, or become adhered together during the dehydration process. This invention provides a solution to this problem. With this invention, recovery of the test organisms is more complete when an excipient (e.g., polyethylene glycol) is employed in the biological indicator in combination with the test organisms.

The prior art methods for producing biological indicators are typically contingent upon multiple components of the biological indicator producing consistent results to maintain predictable resistance levels. A problem in the art relates to the fact that at times unexplained results occur when using biological indicators that can be traced to inconsistencies with respect to the resistance of the biological indicator. With this invention it is possible to control biological indicator resistance levels to meet customer requirements without requiring excessive amounts of screening and selection of manufactured test organisms to find those that show the desired resistance levels. As such, with this invention, it is possible to use fewer batches of test organisms requiring cultivation to meet customer needs for providing biological indicators with desired resistance levels.

In addition, the accuracy with which the resistance of a particular biological indicator is determined is also dependent upon inoculation of the biological indicator with a known population of test organisms, and the ability to show those organisms were successfully recovered from the biological indicator. With this invention, a more efficient means of recovering the test organisms is described allowing for greater accuracy in defining the population of a biological indicator. This invention relates to a biological indicator for use with a sterilization process, comprising a carrier on which an inoculum is placed and dehydrated, the inoculum comprising a test organism in the form of bacterial spores and an effective amount of an excipient (e.g., polyethylene glycol) to enhance recovery of the bacterial spores from the dehydrated inoculum.

In an embodiment, the inoculum further comprises an effective amount of a carbohydrate (e.g., glucose) to reduce the resistance of the biological indicator to the sterilization process.

In an embodiment, this invention relates to a sterilization process, comprising: exposing an article to be sterilized and the above-indicated biological indicator to a sterilant (e.g., steam or vaporous hydrogen peroxide).

In an embodiment, this invention relates to a process for determining the effectiveness of a sterilization process, comprising: exposing an article to be sterilized and the above-indicated biological indicator to a sterilant (e.g., steam or vaporous hydrogen peroxide); and incubating the biological indicator in the presence of a recovery media to determine whether the sterilization process is effective.

### BRIEF DESCRIPTION OF THE DRAWINGS

In the annexed drawings, like parts and like features have like designations.

FIG. 1 is a perspective view of an SCBI which can be used in accordance with the present invention.

FIG. 2 is a cross-sectional view of the SCBI of FIG. 1, taken along line 8-8 in FIG. 1, showing a cap mounted on a container in a first non-activated position;

FIG. 3 is a cross-sectional view of the SCBI of FIG. 1, taken along line 8-8 in FIG. 1, showing the cap mounted on the container in a second activated position.

FIG. 4 is a cross-sectional view of the SCBI of FIG. 1, taken along line 10-10 in FIG. 1, showing the SCBI in a second activated position.

FIG. 5 is a side-top perspective view of a test pack that may be used with an SCBI in accordance with the invention.

FIG. 6 is a cross-sectional view of the test pack illustrated in FIG. 5, taken along line 12-12 of FIG. 5.

FIG. 7 is a top plan view of a test pack that can be used in accordance with the present invention, with an SCBI in a first recessed compartment and a chemical integrator and/or chemical indicator in a second recessed compartment.

### DETAILED DESCRIPTION

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “X and/or Y,” when used in conjunction with open-ended language such as “comprising” can refer, in one embodiment, to X without Y (optionally including elements other than Y); in another embodiment, to Y without X (optionally including elements other than X); in yet another embodiment, to both X and Y (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from any one or more of the elements in the list of elements, but not necessarily including at least one of each and every element specifically listed within the list of elements and not excluding any combination of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of X and Y” (or, equivalently, “at least one of X or Y,” or, equivalently “at least one of X and/or Y”) can refer, in one embodiment, to at least one, optionally including more than one, X, with no Y present (and optionally including elements other than Y); in another embodiment, to at least one, optionally including more than one, Y, with no X present (and optionally including elements other than X); in yet another embodiment, to at least one, optionally including more than one, X, and at least one, optionally including more than one, Y (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

The term “inactivation” of a test organism (e.g., bacterial spores) refers to the loss of ability of the test organism to germinate, outgrow and/or multiply.

The term “log reduction” is a mathematical term to show the number of live test organisms (e.g., bacterial spores) inactivated by contacting the test organisms with a sterilant. A “4 log reduction” means that the number of live test organisms is 10,000 times smaller. A “5 log reduction” means that the number of live test organisms is 100,000 times smaller. A “6 log reduction” means that the number of live test organisms is 1,000,000 times smaller.

The term “sterilization” is often taken to refer to a process wherein a total absence of living test organisms is achieved. However, this term is also used herein to refer to processes that are less rigorous than sterilization processes. These may include, for example, disinfection, sanitization, decontamination, cleaning, and the like. The sterilant used in the sterilization process may comprise steam or vaporous hydrogen peroxide. The sterilization processes provided for herein may be conducted for an effective period of time to achieve at least about a 4 log reduction, or at least about a 5 log reduction, or at least about a 6 log reduction in the number of test organisms capable of germination, outgrowth and/or multiplication.

The term “biological indicator” refers to a microbiological test system which comprises a carrier and test organisms deposited on the carrier. The biological indicator may be used in combination with a process indicator. The biological indicator may comprise a self-contained biological indicator (SCBI).

The term “carrier” refers to a supporting material onto which test organisms may be deposited.

The term “inoculated carrier” refers to a carrier onto which test organisms have been deposited.

The term “test organism” refers to a microorganism in the form of bacterial spores which is used as part of a biological indicator. The test organism is selected from bacterial spores which are more resistant to a sterilization process than the organisms to be destroyed by the sterilization process.

The term “D-value” or “decimal reduction value” refers to the time required to achieve inactivation of 90% of a population of test organisms (also known as a 1 log reduction). The D-value may be expressed in minutes.

The test organisms comprise bacterial spores. These may be spores of the Bacillus or Clostridia genera. The spores may comprise spores of Geobacillus stearothermophilus, Bacillus atrophaeus, Bacillus sphaericus, Bacillus anthracis, Bacillus pumilus, Bacillus coagulans, Clostridium sporogenes, Clostridium difficile, Clostridium botulinum, Bacillus subtilis globigii, Bacillus cereus, Bacillus circulans, or a mixture of two or more thereof. The spores may comprise Geobacillus stearothermophilus spores.

The excipient may comprise polyethylene glycol, polyvinyl pyrolidone, polyvinyl alcohol, dipropylene glycol, polycarboxybetaine methacrylate, polyacrylic acid, polyacrylamide, N-(2-hydroxypropyl) methacrylamide, divinyl ether-maleic anhydride, polyoxazoline, polyphosphate, polyphosphazene, xanthum gum, pectin, a chitosan derivative, dextran, carrageenan, guar gum, cellulose ether, sodium carboxy methyl cellulose, hydroxypropyl cellulose, hydroxypropylmethyl cellulose, hyalurionic acid, albumin, starch, a starch based derivative, or a mixture of two or more thereof.

The polyethylene glycol (PEG) may be linear, branched, star shaped or comb shaped, and may have an average molecular weight in the range from about 300 to about 20,000 grams per mole, or from about 500 to about 20,000, or from about 700 to about 20,000, or from about 1000 to about 20,000, or from about 1000 to about 18,000, or from about 1000 to about 15,000, or from about 2000 to about 15,000, or from about 3000 to about 15,000, or from about 4000 to about 12,000, or from about 6000 to about 10,000, or from about 7000 to about 9000, or about 8000 grams per mole. PEG 8000 may be used.

The carbohydrate may comprise a compound represented by the empirical formula Cₘ(H₂O)ₙ where m and n are numbers. The carbohydrate may comprise carbon, hydrogen and oxygen atoms, with a hydrogen:oxygen ratio of 2:1. The carbohydrate may be a saccharide. The saccharide may be a monosaccharide, disaccharide, or a mixture thereof. The monosaccharides and disaccharides may be referred to as sugars. The monosaccharides may be particularly useful. The monosaccharides may include glucose, fructose, galactose, or a mixture of two or more thereof. Glucose is particularly useful. The disaccharides may comprise maltose, sucrose, lactose, or a mixture of two or more thereof. The carbohydrates may be obtained from sugar cane, sugar beet, corn syrup, and the like.

The biological indicator may comprise a carrier inoculated with an aqueous composition containing the test organisms, an excipient, and optionally a carbohydrate. The aqueous composition is then dehydrated or dried. The carrier may comprise a porous material or a non-porous material. The carrier may comprise a solid material. The carrier may comprise any material that does not dissolve or deteriorate during the sterilization or incubation processes. The carrier may comprise paper, metal, glass, ceramics, plastic, cellulose, or a combination of two or more thereof. The metal may comprise aluminum or steel. The plastic may comprise a polyolefin, polystyrene, polycarbonate, polymethacrylate, polyacrylamide, polyimide, polyester, and the like. The carrier may comprise a film. The carrier may be in the form of a spun or unwoven felt. The carrier may comprise a mat of compressed fibers. The carrier may comprise a porous material made of sintered glass, glass fibers, ceramic, synthetic polymer, or a combination of two or more thereof. The carrier may comprise filter paper or absorbent paper. The carrier may comprise a cellulose pad.

The carrier may be positioned in an SCBI. Alternatively, the carrier may comprise a surface (e.g., a non-porous surface) in the interior of a compartment of an SCBI. The carrier or interior surface of the SCBI may be inoculated with an aqueous composition containing the test organisms, an excipient, and optionally a carbohydrate, and then dehydrated or dried. The SCBI may comprise a capped container with two separate compartments. One of the compartments may contain the inoculated carrier or inoculated interior surface. The other compartment may contain a recovery media. The compartment containing the recovery media may comprise a frangible glass vial containing the recovery media that can be broken just prior to incubation. In use, the SCBI and the articles to be sterilized are exposed to steam during a steam sterilization process. Then following sterilization, the SCBI may be activated to allow for the test organisms to come into contact with the recovery media. The test organisms may then be incubated to determine whether the sterilization process is effective.

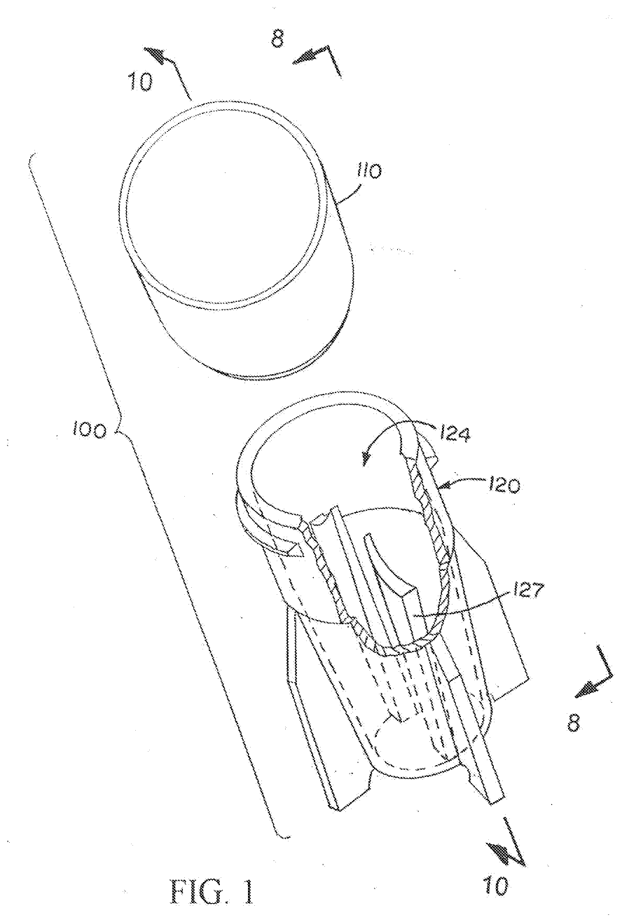

Alternatively, the SCBI may be in the form illustrated in FIGS. 1-4. Referring to FIGS. 1-4, SCBI 100 includes cap 110 which is configured for housing recovery media 140. Recovery media 140 is in the form of a liquid. Cap 110, which is mounted on container 120, includes inner chamber 116. The inner chamber 116 has an opening 115 with a breakable barrier 130 overlying the opening 115. The recovery media 140 is encapsulated within the inner chamber 116. The container 120 has an interior region 124 where test deposit 190 is positioned. The test deposit 190 may be formed by placing an aqueous composition containing the test organisms, an excipient, and optionally a carbohydrate in container 120, and dehydrating or drying the aqueous composition to form test deposit 190.

The concentration of the test organisms (i.e., bacterial spores) in the aqueous composition may range from about 10⁴ to about 10⁸ colony forming units (cfu) per milliliter (ml), or from about 10⁵ to about 10⁷ cfu/ml, or about 10⁶ cfu/ml. The number of test organisms (i.e., bacterial spores) on the carrier may be in the range from about 10⁴ to about 10⁸ cfu/mm², or from about 10⁵ to about 10⁷ cfu/mm², or about 10⁶ cfu/mm². The concentration of the excipient in the aqueous composition may be in the range from about 1 to about 50 milligrams (mg) per milliliter (ml), or from about 1 to about 20 mg/ml, or from about 5 to about 15 mg/ml, or from about 8 to about 12 mg/ml, or about 10 mg/ml. The concentration of the carbohydrate in the aqueous composition may be up to about 20 mg/ml, or in the range from about 0.001 to about 20 mg/ml, or from about 0.01 to about 10 mg/ml, or from about 0.01 to about 5 mg/ml, or from about 0.01 to about 1 mg·ml, or from about 0.05 to about 0.5 mg/ml, or from about 0.08 to about 0.3 mg/ml, or about 0.18 mg/ml. The concentration of the carbohydrate may be from about 1 to about 20 mg/ml, or from about 1 to about 10 mg/ml, or from about 1 to about 5 mg/ml, or from about 1 to about 3 mg/ml, or about 2 mg/ml, or from about 5 to about 20 mg/ml.

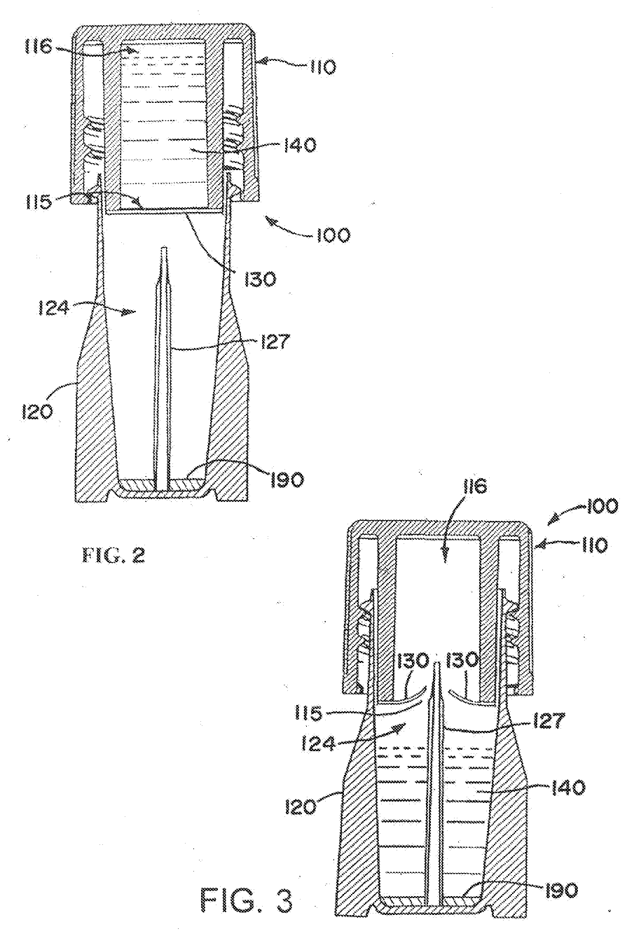

When used in a sterilization process, the cap 110 is held in an open position as illustrated in FIG. 2. The SCBI 100 and items to be sterilized are then subjected to the sterilization process. During the sterilization process, a sterilant (e.g., steam or vaporous hydrogen peroxide) passes through openings between the cap 110 and the container 120 and flows into the interior region 124 where it contacts and acts upon the test deposit 190.

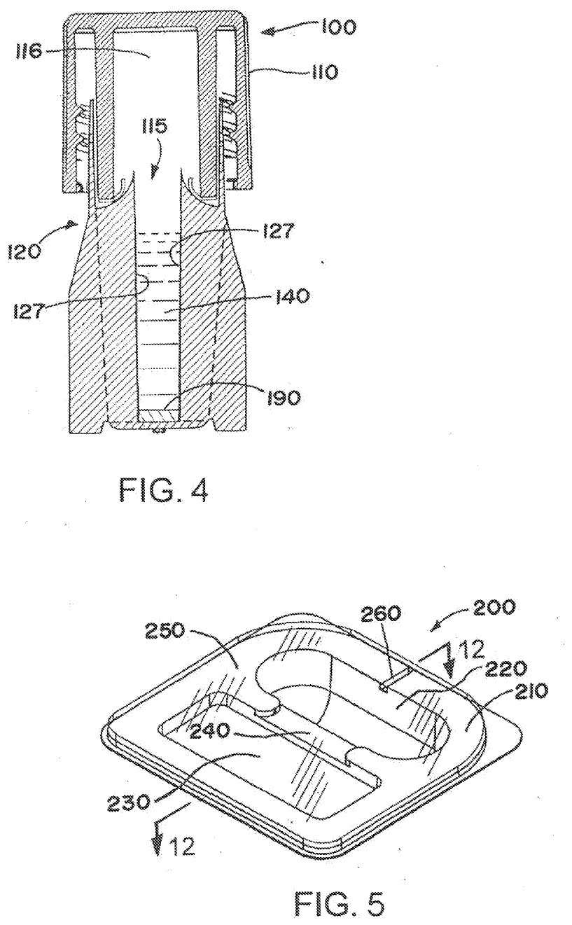

After the sterilization process is complete, the SCBI 100 is activated by screwing the cap 110 downward into a closed position as shown in FIGS. 3 and 4. This results in the breakable barrier 115 being broken by puncture member 127 to form broken barrier 130. (Note that two puncture members may be used in the embodiment shown in FIG. 4). Recovery media 140 then flows into the container 120 in contact with the test deposit 190.

While in container 120, the test deposit 190 and recovery media 140 may be incubated for a sufficient period of time to determine the viability of the spores in the test deposit 190. At the end of the incubation period, the SCBI is evaluated to determine whether any spores survived the sterilization process. If the spores survived the sterilization process, the sterilization process is not considered to have been successful. On the other hand, if the spores are inactivated, then the sterilization process is considered to be successful.

A more detailed description of the SCBI 100 is disclosed in U.S. Pat. No. 8,173,388, which is incorporated herein by reference. It should be noted that SCBI configurations other than those depicted in FIGS. 1-4 may be used.

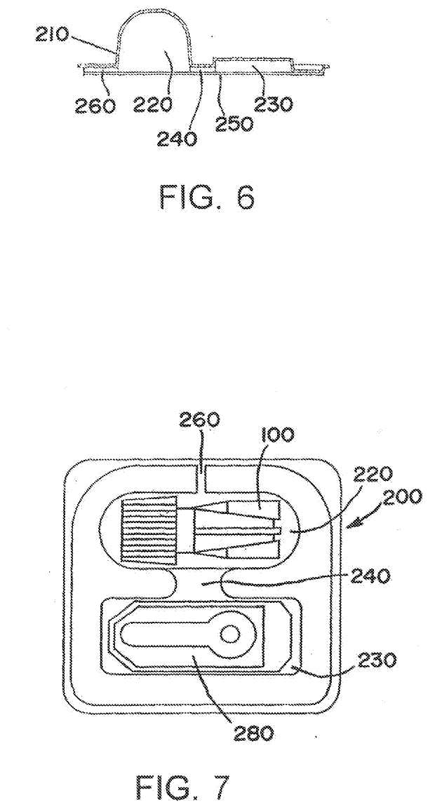

The SCBI 100 may be used with a test pack as depicted in FIGS. 5-7. Referring to FIGS. 5-7, test pack 200 includes base 210 containing recessed compartments 220 and 230. The recessed compartments 220 and 230 are in fluid communication with each other via internal channel 240. Cover 250 is attached to the base 210 and forms a sealed enclosure for the recessed compartments 220 and 230. External channel 260 provides a fluid communication between the sealed enclosure and an external environment. The SCBI 100 is positioned in recessed compartment 220. A chemical integrator and/or a chemical indicator 280 is positioned in recessed compartment 230. The external channel 260 is configured to allow a restricted flow of a sterilant (e.g., steam or vaporous hydrogen peroxide) into the recessed compartments 220 and 230. The base 210 and cover 250 are otherwise impenetrable by the sterilant. The chemical integrator and/or chemical indicator 280 undergoes changes in the position of a leading edge or in color after it has been exposed to a sufficient quantity of sterilant for a sufficient period of time to indicate that the sterilization process has been completed. The sterilant also flows into the SCBI 100 in recessed compartment 220, and contacts the test organisms in the SCBI 100. A more detailed description of test pack 200 is disclosed in U.S. Pat. No. 9,017,944 B2, which is incorporated herein by reference. It should be noted that test pack configurations other than those depicted in FIGS. 5-7 may be used.

Another modification of the above forms of the biological indicator may be a traditional, or a rapid read/fast acting biological indicator. A traditional biological indicator may use a dedicated instrument or incubator to maintain a desired temperature while allowing the test organism to be logarithmically grown in the recovery medium. A rapid read/fast acting biological indicator may be mated to a dedicated instrument (reader) that detects early signals of test organism viability.

The biological indicator may be made by dispersing a desired amount of test organisms (i.e., bacterial spores) in water. The resulting dispersion is spun down, and resuspended in a solution containing the excipient and optionally the carbohydrate prior to being placed on the carrier or in an SCBI container where it is dehydrated or dried. For example, a suspension of Geobacillus stearothermophilus spores in an aqueous carbohydrate solution containing polyethylene glycol, may be prepared to yield a desired number of spores per aliquot for inoculating the carrier. The inoculum may be dispensed and allowed to dry on the carrier. An air flow may be used to dry the inoculum on the carrier, such as, for example, by placing the carrier in a laminar flow-hood to hasten the drying process. The method of drying the inoculum on the carrier may include allowing the inoculum to air dry by leaving it stand under ambient conditions; placing the inoculated carrier in a desiccator containing a desiccant such as calcium chloride, in a temperature and humidity controlled environmental chamber; or placing the inoculated carrier under a stream of dry air, nitrogen or other anhydrous gas. The number of colony forming units (cfu) of the spores supported by the carrier may be in the range from about 10⁴ to about 10⁸ cfu/mm², or from about 10⁵ to about 10⁷ cfu/mm², or about 10⁶ cfu/mm². When ready for use, the SCBI may be placed in a sterilization chamber along with the load to be sterilized and exposed to the sterilant. Once removed from the sterilization chamber the SCBI may be activated causing the release of the recovery media which contacts the test deposit and rehydrates the spores. The SCBI may then be incubated for a prescribed period of time and monitored for survivors.

The biological indicator may be used with any steam or vaporous hydrogen peroxide sterilization process. The biological indicator along with the articles to be sterilized may be exposed to the sterilant during the sterilization process. The sterilization process may be conducted in a sterilization chamber. The sterilization chamber may comprise an autoclave. The temperature for the steam sterilization may typically be in the range from about 121° C. to about 135° C. The biological indicator may be placed in the sterilization chamber in one or more locations where it is difficult for the sterilant to reach to verify that the sterilant is penetrating these locations. Upon completion of the sterilization process, the biological indicator may be incubated in the presence of a growth media to determine whether the sterilization process is effective.

The biological indicator may be used to release loads or validate sterilization chamber functionality in healthcare settings. The biological indicator may also be used to determine if biological indicator waste has been properly decontaminated. In the scientific setting, the biological indicator may be used to validate the functionality of sterilization chambers, release loads of goods, or validate that a process meets required functionality. A valid biological indicator for a given process may require a specific resistance and therefore biological indicator manufacturers may strive to manufacture the biological indicator with targeted resistance characteristics.

To ensure that the biological indicator is appropriate for its intended use, it may be characterized by comparison to a pre-determined resistance. For use in healthcare applications the pre-determined resistance may be established by the U.S. Environmental Protection Agency (EPA) or the U.S. Food and Drug Administration (FDA). For other non-regulated applications it may be established by customer preference. Even in the regulated applications it may be desirable to target a resistance at the low end of the mandated range.

The resistance for the biological indicator may be expressed as its D-value, which quantifies the time required to achieve inactivation of 90% of the population of test organisms. The D-value may be measured in minutes. In the case of steam sterilization, the D-value may be in the range from about 0.01 to about 5 minutes, or from about 0.1 to about 5 minutes.

The resistance of a biological indicator may be targeted to meet customer and/or regulatory requirements. For example, a healthcare biological indicator may be required to meet a targeted D-value for a 121° C. sterilization cycle of 1.5-3.0 minutes. This resistance level may be required by regulatory guidance documents and expected by healthcare consumers. Other standard cycles may vary in the desired or regulated D-value range.

The production of biological indicators may be open to many variables. The specific strain of spore used, the process by which the selected test organism has been cultivated, the scale of cultivation performed, the materials of construction of the carrier and the constituents of the recovery media or other ingredients used in the carrier are all components of the biological indicator that may impact resistance. Minor changes in these components may have significant effects upon the measured resistance characteristics of the biological indicator. This can lead to batches of biological indicators that do not meet the established resistance specifications.

In the prior art, the variability inherent to biological indicator fabrication has led to problems with the manufacturing process and an inability to deliver the desired final product to the customer. Variability in cultivation of the test organism, changes in suppliers of the materials of construction, or lot-to-lot variability in the components of the growth media are a few of the examples of very minor modifications that can lead to unwanted changes in resistance in a biological indicator line that is in production over a period of time.

Additionally, use of specific validated materials of construction in biological indicators allows for limited ability to alter the resistance of a given product to meet customer and regulatory requirements. For example, if a specific product has a D-value of 2.90 minutes in the required conformation, this product will be unlikely to meet the needs of a customer looking for a reduced resistance with a D-value of 1.90 minutes. Both these values may be within the mandated range. To prepare a product having a reduced resistance, the manufacturer may have to cultivate additional lots of test organisms in the hope that the inherent variability of the process would uncontrollably yield the desired lower resistance.

This invention relates to a biological indicator comprising a carrier which is inoculated with an aqueous composition containing a suspension of test organisms, an excipient, and optionally a carbohydrate. The introduction of the carbohydrate to the biological indicator can be used to reduce the resistance of the biological indicator to sterilizations, for example, steam sterilizations. This allows for facilitated production of a biological indicator with a targeted reduced resistance. The introduction of the excipient facilitates interaction of the test deposit with the recovery media.

The biological indicator may be used by subjecting it to the same sterilant and treatment as the articles for which sterile conditions may be sought. The sterilant (e.g., steam or vaporous hydrogen peroxide) may pass into the area where the biological indicator is located thereby exposing the biological indicator to the same sterilization process as the articles being sterilized. Following sterilization, the recovery media may be brought into contact with the biological indicator. The recovery media may be in the form of a liquid. The recovery media may comprise a buffered aqueous solution. Any procedure whereby the biological indicator is brought into contact with the recovery media under conditions which allow for growth of the test organisms, if it still exists, may be used. The recovery media may be present in the sterilization chamber in powder or tablet form and, after sterilization, sterile water may be added such that the biological indicator comes into contact with the recovery media.

The recovery media may comprise one or more nutrient sources. The nutrient source may be used to provide energy for any of the test organisms that survive the sterilization process. Examples of the nutrient sources may include pancreatic digest of casein, tryptic soy broth, enzymatic digest of soybean meal, sucrose, dextrose, yeast extract, L-cystine, a pH indicator such as bromocresol purple, and mixtures of two or more thereof.

A microbial indicator, which changes color or native state, in the presence of viable test organisms may be used with the recovery media. The indicator may be dispersed or solubilized in the recovery fluid and impart an initial color to the media. The growth indicator may also impart a color change in the recovery fluid upon spore growth. Indicators which may be employed include pH-sensitive dye indicators (such as bromothymol blue, bromocresol purple, phenol red, etc. or combinations thereof), oxidation-reduction dye indicators (such as methylene blue, etc.). The use of these microbial indicators may result in a change in color in response to a phenomenon of microorganism metabolism, such as changes in pH, oxidation-reduction potentials, enzymatic activity, as well as other indications of growth.

The recovery media may further comprise one or more pH buffers, one or more neutralizers, one or more agents for maintaining osmotic equilibrium, or a mixture of two or more thereof. The pH buffers may include K₂HPO₄, KH₂PO₄, (NH₄)₂HPO₄, 2,2-Bis(hydroxylmethyl)-2,2′,2″-nitrilothiethanol (Bis Tris), 1, 3-Bis[tris(hydroxymethyl)methylamino] propane (Bis-Tris Propane), 4-(2-Hydroxyethyl)piperazine-ethanesulfonic acid (HEPES), 2-Amino-2-(hydroxymethyl)-1,3-propanediol (Trizma, Tris base), N-[Tris(hydroxymethyl)methyl]glycine (Tricine), Diglycine (Gly-Gly), N,N-Bis(2-hydroxyethyl)glycine (Bicine), N-(2-Acetamido)iminodiacetic acid (ADA), N-(2-Acetamido)-2-aminoethanesulfonic acid (aces), 1,4-Piperazinediethanesulfonic acid (PIPES), β-Hydroxy-4-morpholinepropanesulfonic acid (MOPSO), N,N-Bis(2-hydroxyethyl)-2-aminoethanesulfonic acid (BES), 3-(N-Morpholino)propanesulfonic acid (MOPS), 2-[(2-Hydroxy-1,1-bis(hydroxylmethyl)ethyl)amino]ethanesulfonic acid (TES), 3-(N,N-Bis[2-hydroxyethyl]amino)-2-hydroxypropanesulfonic acid (DIPSO), 4-(N-Morpholino)butanesulfonic acid (MOBS), 2-Hydroxy-3-[tris(hydroxymethyl)methylamino]-1-propanesulfonic acid (TAPSO), 4-(2-Hydroxyethyl)piperazine-1-(2-hydroxypropanesulfonic acid hydrate (HEPPSO), Piperazine-1,4-bis(2-hydroxypropanesulfonic acid) dihydrate (POPSO), 4-(2-Hydroxyethyl)-1-piperazine propanesulfonic acid (EPPS), N-(2-Hydroxyethyl)piperazine-N′-(4-butanesulfonic acid) (HEPBS), [(2-Hydroxy-1,1-bis(hydroxymethyl)ethyl)amino]-1-propanesulfonic acid (TAPS), 2-Amino-2-methyl-1,3-propanediol (AMPD), N-tris(Hydroxymethyl)methyl-4-aminobutanesulfonic acid (TABS), N-(1,1-Dimethyl-2-hydroxyethyl)-3-amino-2-hydroxypropanesulfonic acid (AMPSO), 2-(Cyclohexylamino)ethanesulfonic acid (CHES), 3-(Cyclohexylamino)-2-hydroxyl-1-propanesulfonic acid (CAPSO), 2-Amino-2-methyl-1-propanol (AMP), 3-(Cyclohexylamino)-1-propanesulfonic acid (CAPS), 4-(Cyclohexylamino)-1-butanesulfonic acid (CABS), 2-(N-Morpholino)ethanesulfonic acid hydrate (MES), N-(2-Acetamido)-2-aminoethanesulfonic acid (ACES), and mixtures of two or more thereof.

The neutralizers may include but are not limited to sodium thioglycollate, sodium thiosulfate, catalase, sodium bisulfate, sodium bisulfite lecithin, polysorbate 20, polysorbate 80, calcium bicarbonate, and mixtures of two or more thereof.

The agents for maintaining osmotic equilibrium may include sodium salt, potassium salts, magnesium salts, manganese salts, calcium salts, metallic salts, sodium chloride, potassium chloride, magnesium sulfate, iron chloride, and mixtures of two or more thereof.

The recovery media may comprise an aqueous composition comprising: water; from about 0.01 to about 100 grams per liter (gift or from about 0.1 to about 50 g/l, of one or more nutrient sources; from about 1.0×10⁻⁵ to about 10 g/l, or from about 1.0×10⁻⁴ to about 1.0 g/l of one or more microbial growth indicators; up to about 5000 g/l, or from about 0.001 to about 5000 g/l, or from about 0.1 to about 1000 g/l, of one or more pH buffers; up to about 100 g/l, or from about 0.01 to about 100 g/l, or from about 0.1 to about 50 g/l, of one or more neutralizers; up to about 50 g/l, or from about 0.1 to about 50 g/l, or from about 0.1 to about 25 g/l, of one or more agents for maintaining osmotic equilibrium.

The recovery media may contain one or more enzyme substrates to detect the presence of any enzymes produced by the test organism. Where the enzyme whose activity is to be detected is alpha-D-glucosidase, chymotrypsin, or fatty acid esterase, e.g., from Geobacillus stearothermophilus, an enzyme substrate that may be used is 4-methylumbelliferyl-alpha-D-glucoside, 7-glutarylphenylalanine-7-amido-4-methyl coumarin, or 4-methylumbelliferyl heptanoate. Where the enzyme whose activity is to be detected is alpha-L-arabinofuranosidase, e.g., derived from Bacillus subtilis, an enzyme substrate that may be used is 4-methylumbelliferyl-alpha-L-arabinofuranoside. Where the enzyme whose activity is to be detected is beta-D-glucosidase, e.g., derived from Bacillus subtilis, an enzyme substrate that may be used is 4-methylumbelliferyl-beta-D-glucoside. Additional enzyme substrates that may be used are disclosed in U.S. Pat. No. 8,043,845 B2, which is incorporated herein by reference.

The recovery media may contain one or more amino acids (e.g., L-Glutamic acid, L-Histadine, L-Isoleucine, L-Methioine, L-Valine), one or more vitamins (e.g., D-Biotin, Thiamine HCl, Nicotinic Acid), one or more metals and/or minerals (e.g., FeCl₃, CaCl₂), ZnSO₄, MnCl, NH₄Cl, NaCl, MgSO₄, K₂HPO₄, KH₂PO₄), 4-methylumbelliferyl-α-D-glucopyranoside (MUD), dimethylsulfoxide (DMSO), and Bromocresol Purple.

### Example 1

Polyethylene glycol with a molecular weight of 8000 (PEG 8000) is used as an excipient to affect a greater recovery of certain spore cultures in dehydrated SCBIs. The spores are Wild-Type lot 20 spores (Geobacillus stearothermophilus 7953) Smith.

A sample of 42 ml of Wild-type spores is vortexed for several minutes to homogenize the cultures. This sample is designated as WT-20. An aliquot of 20 ml of WT-20 is transferred by serial pipette to a second container. 200 mg of PEG 8000 are added to the 20 ml aliquot to achieve a 1% w/v PEG/WT-20 culture, designated WT-20PEG.

WT-20 and WT-20 PEG are inoculated into 144 SCBI vials corresponding to SCBI 100 shown in FIGS. 1-4.

For Wet Vial concentrations WT-20 and WT-20PEG, three still-wet SCBI vials are recovered using pure water and serially diluted at a 10⁻⁴ dilution. 20 μl of the final dilution is pour-plated in triplicate, tempered, and incubated for 2 days at 57° C.

For Dry Vial concentrations, the method is similar to the Wet Vial method, except that greater effort is utilized in attempting to remove spores via pipetting, typically using more than three attempts per vial, and 400 μl of the final dilution are pour-plated.

A 121° C. D-value is performed with exposure times of 8, 11, 12, 13, and 18 minutes. Ten SCBIs are exposed from each culture simultaneously to maintain uniform test conditions. Each exposed SCBI is transferred to bromocresol purple tubes via pipette.

The signal from both cultures is examined to detect any influence from PEG by activating four SCBI vials from each culture, running the vials on a reader for 40 minutes, followed by analysis. The results are shown in Tables 1 and 2.

TABLE 1
Wet Vial (CFU)

| WT-20 | Results | Average | WT-20PEG | Results | Average |
|---|---|---|---|---|---|
| Vial 1 | 56, 50, 52 | 53 | Vial 1 | 47, 50, 40 | 46 |
| Vial 2 | 39, 54, 50 | 48 | Vial 2 | 45, 43, 45 | 44 |
| Vial 3 | 44, 32 ,45 | 40 | Vial 3 | 43, 39, 52 | 45 |
| 2.35E6/Vial | | 47 | 2.25E6/Vial | | 45 |

TABLE 2
Dry Vial (CFU)

| WT-20 | Results | Average | WT-20PEG | Results | Average |
|---|---|---|---|---|---|
| Vial 1 | 14, 14, 18 | 15 | Vial 1 | 94, 92, 83 | 90 |
| Vial 2 | 20, 17, 10 | 16 | Vial 2 | 89, 76, 83 | 83 |
| Vial 3 | 11, 14, 10 | 12 | Vial 3 | 91, 99, 93 | 94 |
| 3.5E5/Vial | | 14 | 2.23E6/Vial | | 89 |

The tables above show the number of CFUs (colony forming units) recovered per condition during spore recovery experiments. Table 1, labeled as Wet Vial (CFU) is a control for the experiment for the two conditions: spores containing PEG and spores without the PEG. These are inoculated into an SCBI, then recovered to show that there is no significant difference between spores with and without PEG.

Table 2, labeled as Dry Vial (CFU), discloses test results wherein the vials are inoculated identically as above, but allowed to dry. If the PEG has no effect on recovery, then both conditions with and without PEG should not have significant recovery differences from each other. However, if PEG has either a positive or negative effect on dry spore recovery, it would be apparent in the number of CFUs recovered. If spores with PEG have counts more in line with the Wet Vial Table 1, then PEG has a positive effect on recovery.

Each test is initiated from the same spore lot containing over two million spores per 20 ul aliquot. For both Wet Vial conditions, identical averages (in microbiological terms) are observed. For Dry Vial with PEG, identical results (2× the volume are plated for these, so the average would be 44.5) are observed. The dry, no PEG result shows only an average of 14 (half again, would be 7) which is equivalent to only three-hundred fifty thousand spores recovered. Since each vial contains over two million spores, it is determined that drying exacerbates spore recovery, while the addition of PEG solves the dry recovery issue.

### Example 2

A standard lot of Geobacillus stearothermophilus spores is suspended in an aqueous solution containing 0.18 mg/ml of glucose and 10 mg/ml of PEG 8000 to form an inoculum. The approximate concentration of spores in the inoculum is 10⁶ cfu per milliliter. The inoculum is placed in a compartment of an SCBI and dehydrated to form a test deposit. The SCBI corresponds to SCBI 100 shown in FIGS. 1-4. The compartment corresponds to container 120 shown in FIGS. 1-4. The test deposit corresponds to test deposit 190 shown in FIGS. 2-4. The presence of glucose in the test deposit reduces the resistance of the test deposit to steam during a steam sterilization. The PEG 8000 affects the recoverability of the spores after the steam sterilization is completed. This provides for a more accurate D-value for the SCBI.

While the invention has been explained in relation to various embodiments, it is to be understood that various modifications thereof will become apparent to those skilled in the art upon reading the specification. Therefore, it is to be understood that the invention disclosed herein includes any such modifications that may fall within the scope of the appended claims.

1. A biological indicator for use with a sterilization process, comprising: a carrier on which an inoculum is placed and dehydrated, the inoculum comprising a test organism in the form of bacterial spores and an effective amount of an excipient to enhance recovery of the bacterial spores from the dehydrated inoculum, and wherein the inoculum further comprises an effective amount of a carbohydrate to reduce the resistance of the biological indicator to the sterilization process.

2. (canceled)

3. The biological indicator of claim 1 wherein the carrier comprises a porous material.

4. The biological indicator of claim 1 wherein the carrier comprises a non-porous material.

5. The biological indicator of claim 1 wherein the carrier comprises metal, glass, ceramic, plastic, cellulose, paper, or a combination of two or more thereof.

6. The biological indicator of claim 1 wherein the carrier comprises an interior surface of a self-contained biological indicator.

7. The biological indicator of claim 1 wherein the carrier comprises an interior surface of a first compartment of a self-contained biological indicator, the first compartment being adapted to permit the spores to be brought into contact with a sterilant during the sterilization process, and the self-contained biological indicator further comprising a second compartment containing a recovery media, the second compartment being adapted to maintain the recovery media separate from the spores during the sterilization process, and the second compartment being adapted to permit the recovery media to contact the spores after the sterilization process is completed.

8. The biological indicator of claim 1 wherein the carrier comprises a support positioned in a first compartment of a self-contained biological indicator, the first compartment being adapted to permit the spores to be brought into contact with a sterilant during the sterilization process, and the self-contained biological indicator further comprising a second compartment containing a recovery media, the second compartment being adapted to maintain the recovery media separate from the spores during the sterilization process, and the second compartment being adapted to permit the recovery media to contact the spores after the sterilization process is completed.

9. The biological indicator of claim 7 wherein the self-contained biological indicator is positioned in a test pack.

10. The biological indicator claim 1 wherein the spores are of the Bacillus or Clostridia genera.

11. The biological indicator claim 1 wherein the spores are Geobacillus stearothermophilus, Bacillus atrophaeus, Bacillus sphaericus, Bacillus anthracis, Bacillus pumilus, Bacillus coagulans, Clostridium sporogenes, Clostridium difficile, Clostridium botulinum, Bacillus subtilis globigii, Bacillus cereus, Bacillus circulans, or a mixture of two or more thereof.

12. The biological indicator claim 1 wherein the spores are Geobacillus stearothermophilus.

13. The biological indicator claim 1 wherein the excipient is selected from polyethylene glycol, polyvinyl pyrolidone, polyvinyl alcohol, dipropylene glycol, polycarboxybetaine methacrylate, polyacrylic acid, polyacrylamide, N-(2-hydroxypropyl) methacrylamide, divinyl ether-maleic anhydride, polyoxazoline, polyphosphate, polyphosphazene, xanthum gum, pectin, chitosan derivative, dextran, carrageenan, Guar gum, cellulose ether, sodium carboxy methyl cellulose, hydroxypropyl cellulose, hydroxypropylmethyl cellulose, hyalurionic acid, albumin, starch, starch based derivative, or a mixture of two or more thereof.

14. The biological indicator claim 1 wherein the excipient comprises polyethylene glycol.

15. The biological indicator of claim 14 wherein the polyethylene glycol has an average molecular weight in the range from about 4000 to about 12,000 grams per mole.

16. The biological indicator of claim 1 wherein the carbohydrate comprises a saccharide.

17. The biological indicator of claim 1 wherein the carbohydrate comprises a monosaccharide, disaccharide, oligosaccharide, or a mixture of two or more thereof.

18. The biological indicator of claim 1 wherein the carbohydrate comprises xylose, glucose, fructose, galactose, arabinose, ribulose, mannose or a mixture of two or more thereof.

19. The biological indicator of claim 1 wherein the carbohydrate comprises triose, tetrose, pentose, hexose, heptose, octose, nonose, or a mixture of two or more thereof.

20. The biological indicator of claim 1 wherein the carbohydrate comprises glucose.

21. The biological indicator of claim 1 wherein the spores are Geobacillus stearothermophilus spores, the excipient is polyethylene glycol with a molecular weight in the range from about 4000 to about 12,000 grams per mole, and the carbohydrate is glucose.

22. The biological indicator of claim 21 wherein the concentration of the polyethylene glycol in the inoculum is in the range from about 1 to about 20 mg/ml, and the concentration of the glucose in the inoculum is in the range from about 0.001 to about 1 mg/ml.

23. The biological indicator of claim 1 wherein the concentration of the spores in the inoculum is in the range from about 10⁴ to about 10⁸ cfu per milliliter.

24. The biological indicator of claim 1 wherein the number of spores on the carrier is in the range from about 10⁴ to about 10⁸ cfu/mm².

25. The biological indicator of claim 1 wherein the biological indicator has a D-value in the range from about 0.01 to about 5 minutes.

26. A sterilization process, comprising:
exposing an article to be sterilized and the biological indicator of claim 1 to a sterilant.

27. The process of claim 26 wherein the sterilant is steam.

28. The process of claim 26 wherein the sterilant is vaporous hydrogen peroxide.

29. A process for determining the effectiveness of a sterilization process, comprising:
exposing an article to be sterilized and the biological indicator of claim 1 to a sterilant; and
incubating the biological indicator to determine whether the sterilization process is effective.

30. The process of claim 29 wherein the sterilant is steam.

31. The process of claim 29 wherein the sterilant is vaporous hydrogen peroxide.
